(19) United States
(12) Patent Application Publication
(10) Pub. No.: US 2016/0025708 A1
(43) Pub. Date: Jan. 28, 2016

(54) METHOD FOR TARGETTING GROWTH AND DEATH OF NEOPLASTIC CELLS BY BURSTS OF ENERGIES FROM CELLULAR ENERGY EMISSIONS

(71) Applicants: Mahmood Mirhoseini, Germantown, WI (US); Mary Cayton Mirhoseini, Germantown, WI (US); Aria Manasheri, Germantown, WI (US)

(72) Inventors: Mahmood Mirhoseini, Germantown, WI (US); Mary Cayton Mirhoseini, Germantown, WI (US); Aria Manasheri, Germantown, WI (US)

(21) Appl. No.: 14/873,204

(22) Filed: Oct. 2, 2015

(51) Int. Cl. G01N 33/50; C12N 13/00

(52) U.S. Cl. CPC G01N 33/5091; C12N 13/00

(57) ABSTRACT

The embodiments herein disclose a non-invasive method of using bursts of energies/electromagnetic field energies from cells to reduce or arrest the growth rate, proliferation of cancer cells/neoplastic cells. The energy from cells induces apoptosis in cancer cells, without harming normal cells beyond their physiologic threshold of survival are provided. The embodiments herein disclose a method for treatment of cancer/neoplastic cell in human or animals within the context of cancer therapeutics. A cell culture plate is incubated. This plate serves as the source of bursts energies/electromagnetic field energies. Further with a device the bursts of energies are targeted to another plate having cells for one week. After one week the microscopic examination is done. The rate of growth of cell is six to seven pulsatile cells per square centimetre. The energy from cells kills cancer cells, induce apoptosis, stimulate growth phase in cell culture and enables harmonics therapy.

### BACKGROUND

1. Technical Field

The embodiments herein generally relates to the field of electromagnetic waves which fall into ultraviolet band. The embodiments herein particularly relate to targeting neoplastic cells using electromagnetic waves/energy. The embodiment herein more particularly relate to the harnessing the electromagnetic energy from the biological/living cells of an organism. The embodiments herein also relate to targeting the electromagnetic energy from the living cells for targeting a cell growth and a cell death of neoplastic cells.

2. Description of the Related Art

The animal or plant cells are complex structures anatomically and physiologically. The cell boundary in the living cell is the cell membrane. The cell membrane has two layers of lipophilic material. The cell inside the cell membrane is filled with cytosol. There are many cellular structures within cytosol. All the cells have nucleus and nucleolus. The nucleus and nucleolus consist of the genetic material, specifically DNA or RNA. In addition to nucleus and nucleolu, there are multiple organelles within the cells. The most important structure in any cell is mitochondria. Mitochondrion is termed as “power house of a cell”. Destruction of mitochondria causes the death of the cell. Most of the energy emitted by the cell is generated by mitochondria.

The membrane potential of the cell is a sum of all energy within the cell. In case of cardiac cells, the membrane potential is ±70 millivolts, depending on a flux of Na and potassium across the membrane. The change of action potential causes a contraction of heart muscle. All the cells do not have a charge of ±70 millivolts in their membranes. The cell having a charge of this range is CD34 stem cell produced by bone marrow after special culture procedure.

The animal and plant cells communicate with each other through electro-magnetic fields which falls into ultraviolet (UV) band. These signals are weak, but release bursts of energies at certain intervals. These bursts of energies are detectable. The bursts of energies are binary in nature, similar to computer. These signals travel several centimeters and these signals are amplified for transmission for longer distances.

The cell signals are different from the action potential of cell membranes, which in cardiac cells (myocytes) is ±70 milli-volts, depending on the influx of ions of K+ (potassium) or Na (sodium) through the cell membrane. Binary cell signals are produced by an oxidation of acids within the cells. These cell signals are different from the membrane action potential. These binary signals or cell signals are called excitons. The excitons are strongly bound to the electro-magnetic field.

The electric fields of endogenous origin have been measured outside the periphery of cultured cells, within multiple tissues and cell types of developing embryos and at the borders of healing and regenerating tissues. The electrically charged and charge dependent molecules of cells and tissues are naturally inherent to the biological systems and assist in defining their electro-physiological and functional properties thereby permitting them to sub-regulate and interact with their associated molecules and related biological systems.

At the molecular level of all cells, tissues and organs, the physiological and biochemical process of directing the cell survival, growth, proliferation, and functions such as programmed cell death, require a complex series of fundamental alterations and modifications. The alterations and modifications in the cells are electrostatic bonding interactions within a given bio-regulatory systems. The electrostatic bonding/charge dependent cell bio-regulatory systems are naturally inherent within all living cells and tissues. Certain exogenously applied electromagnetic fields of low energy have been demonstrated to alter the cell membrane signaling systems, cell membrane potential, oxidative/reductive process and rates, DNA transcription, thermodynamic and kinetic driven protein folding, ion drift and collision rate, immune cell response and enzyme activity when applied to biological system.

The electromagnetic fields of low energy have been used therapeutically for several years or more to stimulate a bone growth and a repair as well as healing of other various tissues in humans and animals by making use of this phenomenon.

Neoplasia (meaning-new growth) is a term that is used to define the development of a cell that has altered in such a way that its growth exceeds. The growth is not coordinated within neoplastic cells, when compared to the normal tissues. An unchecked growth or multiplication of a neoplastic cell forms a mass of cells called as neoplasm. When a neoplasm reaches a clearly recognizable mass, it is usually referred to as a tumour. A cancer is a malignant neoplasm or tumour.

Neoplasm is an abnormal growth of tissue, and is commonly referred to as a tumor or tumour when a mass is formed. This abnormal growth (neoplasia) usually but not always forms a mass.

The World Health Organization (WHO) classifies neoplasms into four main groups: benign neoplasms, in situ neoplasms, malignant neoplasms, and neoplasms of uncertain or unknown behavior. Malignant neoplasms are also simply known as cancers.

Prior to the abnormal growth of tissue, as neoplasia, cells often undergo an abnormal pattern of growth, such as metaplasia or dysplasia. However, metaplasia or dysplasia does not always progress to neoplasia.

Neoplastic tumors are often heterogeneous and contain more than one type of cell. But their initiation and continued growth is usually dependent on a single population of neoplastic cells. These cells are presumed to be clonal thereby indicating that they are derived from the same cell, and all carry the same genetic or epigenetic anomaly (which is evidence for clonality). For lymphoid neoplasms, such as lymphoma and leukemia, clonality is proved by the amplification of a single rearrangement of their immunoglobulin gene (for B cell lesions) or T-cell receptor gene (for T cell lesions). The demonstration of clonality is now considered to be necessary to identify a lymphoid cell proliferation as neoplastic.

A neoplasm is caused by an abnormal proliferation of tissues, which is caused by genetic mutations. Not all types of neoplasms cause a tumorous overgrowth of tissue, however (such as leukemia or carcinoma in situ) forms a tumorous overgrowth of tissue.

There are many causes of neoplasm. The causes are known as DNA damage, DNA mutation, exposure to chemical agents, oncogenic virus, chromosomal anomaly and exposure to radiations.

DNA damage is considered to be the primary underlying cause of malignant neoplasms known as cancers. DNA damage is very common. The central features of DNA damage, epigenetic alterations and deficient DNA repair in progression to cancer are the sequence of events in the formation of neoplasm. Naturally occurring DNA damages (mostly due to cellular metabolism and the properties of DNA in water at body temperatures) occur at a rate of more than 60,000 new damages, on average, per human cell, per day. Additional DNA damages arise from an exposure to exogenous agents. Tobacco smoke causes an increased exogenous DNA damage, and these DNA damages are the likely cause of lung cancer due to smoking. UV light from solar radiation causes DNA damage that is important in melanoma. Helicobacter pylori infection produces high levels of reactive oxygen species that damage DNA and contributes to gastric cancer.

DNA mutation is the basis for cell transformation in neoplasm or cancer-development. The disrepair of DNA is the main source of DNA mutations in somatic cells. The surviving rate of a cell after DNA disrepair is low. The accumulation of DNA disrepairs (mutations) take place in the cells and their offspring cells. The offspring cells proliferate with the DNA having disrepair. The cell transformation is a slow and long process, because the accumulation of DNA mutations needs to proceed over many generations of cells.

The terms “field cancerization” and “field defect” have been used to describe pre-malignant tissue in which new cancers are likely to arise. Field defects are important in progression to cancer. The epigenetic alterations present in tumors occur in pre-neoplastic field defects.

The cells transformed into neoplastic cells have the following characteristics: Loss in cells apoptosis or cell death activity, loss in the cell cycle control, loss in the ability of tumor suppression, abnormal angiogenesis, immune evasion, impaired DNA repair.

For the treatment of neoplasia the following methods are used. the methods include surgery, radiation therapy, chemotherapy, pharmacogenetic considerations, immunological therapy.

Surgery is the most common procedure to treat tumors, neoplastic tumors or carcinoid tumors. Surgery is used to treat primary neoplasm and nearby lymph nodes where the tumor has spread. The surgery is commonly performed on bowel/colorectal tumors, liver tumors, appendix tumor. The surgery may not help to completely remove the tumor cells/tissues. Further surgery is combined with radiation therapy and chemotherapy.

Radiation therapy is generally used to treat carcinoid tumors. The radiation therapy helps people who cannot have surgery and radiation relieve from pain, when the cancer has been spread.

Chemotherapy is generally prescribed for all tumors. But chemotherapy has many side effects such as anemia, weakened immune system, nervous and muscular weakness, bowel/digestive problems, pale skin, hair loss, adverse effect on sexual and reproduction system, damage to kidney and liver.

In the context of cancer therapeutics, there are physiologic differences between the normal cells and neoplastic cells/cancer cells. The neoplastic cells/cancer cells have different physiologic differences/sensitivity than normal healthy cells to the electromagnetic field applications. The methods and applications for electromagnetic field causes inductive coupling to cancer cells and cancerous tumor tissues are utilized to adversely affect the cancer cell's bio-regulatory growth, proliferation and survival systems. The electromagnetic field affects the cancer cells without harming normal cells and tissues. The embodiments herein are directed specifically toward adversely altering the bio-regulatory electrical energies of cancer cells, specifically, the bio-regulatory electrical energies that are involved in the physiologic processes of cancer cell growth, proliferation, and survival.

Beloussov and van Wijik first investigated and coined the term “biophotonics”. The term “biophotonics” refer to waves attributed to optical and ultraviolet photons emitted by the living bio-systems. The process for production was thought to be different from chemical luminescence. The measurement of these waves was accomplished by low noise electronic photo detectors. Biophoton production is now observed in many living materials. The spectra of biophotons is within and ultraviolet range and is different from spectra of systems with temperature about 300° K.

The mechanism of biophoton production is stipulated by biochemical reactions. When the atoms of proteins and acids are oxygen are oxidized, then the rate of biophoton production is low, and affect the other bio-systems.

When the cells are in the state of growth then the radiation reaches to several centimeters. The radiations increase the rate of cell division (mitosis) upto 30% higher to the standard values.

Hence there is a need for a method for targeting neoplastic cells without any side effects. Also there is a need for a simple method for targeting the neoplastic cells with electromagnetic radiation.

The above mentioned shortcomings, disadvantages and problems are addressed herein and which will be understood by reading and studying the following specification.

### OBJECTIVES OF THE EMBODIMENTS

The primary objective of the embodiment herein is to analyze the source of action potential of progenitor cells and precise measurement of action potential of the progenitor cells along with cell organelles.

Another object of the embodiment herein is to measure the action potential in CD4 cells or progenitor cells of bone marrow and to establish a relation of action potential and cell membrane.

Yet another object of the embodiment herein is to analyze the action potential and cell signals in aging cells, in apoptotic cell and stimulated growth, mitotic phase.

Yet another object of the embodiment herein is to arrest the cell signal production by producing a negative harmonic (harmonics) for destruction of the cells (cancer cells/neoplastic cells).

Yet another object of the embodiment herein is to analyze whether the action potential in myocytes and the progenitor cells are similar or different.

Yet another object of the embodiment herein is to test different probes, micro or nano voltmeters and oscilloscopes to measure the action potential of cell organelles such as mitochondria, golgi apparatus.

Yet another object of the embodiment herein is to compare the action potential of different cell organelles in different electrolytic media.

Yet another object of the embodiment herein is to stimulate the growth phase in a culture media, bioreactors comprising young cells/tissues and old cells/tissues to evaluate the effect of aging process.

Yet another object of the embodiment herein is to measure the action potential signals in the mitotic phase of cells growth.

Yet another object of the embodiment herein is to measure the action potential in apoptotic phase and cell death phase (as in case of ischemic cells).

Yet another object of the embodiment herein is to determine the harmonics of the binary cell signals and reverse harmonics of the cells.

Yet another object of the embodiment herein is to analyze the effect of the photodynamic therapy (PDT) and evaluate the signals and the effect of reverse harmonics.

Yet another object of the embodiment herein is to analyze the effect of cell signals on neoplastic cells/cancer cells.

These objects and the other advantages of the embodiments herein will become readily apparent from the following detailed description taken in conjunction with the accompanying drawings.

### SUMMARY

The various embodiments herein provide a method for targeting neoplastic cells without any side effects. Further the embodiments provide a simple method for targeting the neoplastic cells with electromagnetic radiation.

According to one embodiment herein, the method for stimulating a cell culture for growth with bursts of energies/cellular signals of cells, comprises of the following steps. The CD34 cells are cultured in three different culture plates comprising culture medium by following a standard culture protocol. The first culture plate is labelled as a control plate. The control plate comprises the CD 34 cells in culture medium. The first culture plate is incubated at 37° C. for one week. The first culture plate is not exposed to any bursts of energies/cellular signals of cells.

The second culture plate comprising CD 34 cells in the culture medium is subjected to red light. The CD34 cells are stimulated by the red light constantly for one week. The second culture plate is incubated at 37° C. for one week.

The third culture plate comprising CD 34 cells in a culture medium is subjected to plurality of bursts of energies/cellular signals of the CD34 cells in the second culture plate with device. The plurality of bursts of energies/cellular signals of the CD34 cells is detected and amplified by photo multiplier. The third culture plate is incubated at 37° C. for one week. The three culture plates comprising CD 34 cells are analyzed microscopically after one week.

According to one embodiment herein, the control culture plate illustrates 1-2 CD34 cells per square centimeter pulsatile. The second culture plate illustrates 3-4 CD34 cells per square centimeter pulsatile. The third culture plate illustrates 6-7 CD34 cells per square centimeter pulsatile. The photonic and electromagnetic waves increase a growth in number of cells.

According to one embodiment herein, the plurality of bursts of energies/cellular signals of the cells production is stipulated by biochemical reactions in the cells. The biophotons or bursts of energies/cellular signals of cells are produced by oxidization of proteins and acids in presence of oxygen in cell.

According to one embodiment herein, the cells producing bursts of energies/cellular signals of cells are in a state of growth. The bursts of energies/cellular signals of cells reach to nearby cells. The bursts of energies/cellular signals of cells increase a rate of division (mitosis) to a range. The range is 0-30%.

According to one embodiment herein, the transfer of the bursts of energies/cellular signals are binary in nature.

According to one embodiment herein, the bursts of energy are a summation of interior signals and membrane signals of cell organelles. The cell organelles produce action potential. The bursts of energy fall within an ultraviolet band of electromagnetic spectrum.

According to one embodiment herein, the method for evaluating aging in different cells with bursts of energies/cellular signals comprises the following steps. The epithelial cells from foreskin of circumcised new born infant are obtained. The cells from foreskin of the infant are cultured in laboratory following a standard protocol. The cells from upper thigh tissue of 80 year old individual are obtained. The cells from upper thigh tissue of 80 year old individual are cultured in laboratory following a standard protocol. The bursts of photons emitted from the cell culture of newborn foreskin tissue cells and upper thigh tissue of an individual are analyzed. The bursts of energies/cellular signals emitted from the new born foreskin tissue cells are more than that of the bursts of energies/cellular signals emitted from upper thigh tissue of an individual.

According to one embodiment herein, the method of analyzing photonic signals/bursts of energies/cellular signals emitted from apoptotic/cell undergoing cell death, comprises the following steps. Two endobronchial cancer cell sample are obtained from a patient. The first sample of the endobronchial cancer cell is obtained when the patient is not administered any chemical agent or a drug. The cells are cultured according to a standard protocol. The second sample of the endobronchial cancer cell are obtained after administering the patient with a photoferin II at a concentration of 2.5 mgm/Kg body weight. The endobronchial cancer cells are taken after 48 hours from a time of treating with a 630 nm laser light. The sample-2 cells undergo cell death/apoptosis.

According to one embodiment herein, the first sample emits photonic signals/bursts of energies/cellular signals. The second sample emits erratic signal or noise. The second sample cells indicate that the cells undergoing cell death do not produce photonic signals/bursts of energies/cellular signals.

According to one embodiment herein, the mechanism of biophoton production is stipulated by biochemical reactions in the cells. When the atoms of proteins and acids are oxidized in presence of oxygen the biophoton's are produced, and affect the other bio-systems.

According to one embodiment herein, when the cells are in the state of growth then the radiation emitted by the cells reaches to several centimeters. The radiations increase the rate of cell division (mitosis) upto 30% higher to the standard values.

According to one embodiment herein, the phenomenon of mitotic energy from a cell emits radiation to a level of 10⁴ more intense for cellular communication or bio-system communication in a state of apoptosis or stress. The mitogenic effect cannot be described within the standard framework of cellular and biological systems; it is very similar to information exchange between two distant computers by binary encoded massages. The analysis of the signal exchange is done with a photomultiplier.

According to one embodiment herein, optical and ultraviolet excitation is released within the cell medium. The optical and ultraviolet excitations released are quasi-particles called exciton. These excitons are spread freely within the whole volume of the culture media. The excitations are strongly coupled with the electromagnetic field, therefore the excitons are effectively produced during photo absorption by the plants. The inverse process results in photon emission from biological medium.

According to one embodiment herein, excitons play an important role in the transfer of energy inside the cell, particularly during photosynthesis in plants and bacteria. Photon production by excitons is apparently non liner, for long distances of travel these photons should have solitonic properties. Exciton exchanges constitute effective signaling and affect plant growth.

According to one embodiment herein, the rate of biophoton emission is low at about 10 photons/cm²/second from the surface of a large bio system. In a non-coherent field of photons, it is called a stochastic assembly of photons. In this case a single photon or narrow bunch of photons, detected by a bio system as a single click or one bit of information, is analogous to a standard photon detection device.

According to one embodiment herein, the same approach is applicable for exciton signaling produced or absorbed in a bio system. Under these conditions the exciton signaling between two parts of the same bio system and photo signaling between two distant bio systems is similar.

According to one embodiment herein, the signals which control cell mitosis and cellular functions are similar to the standard binary encoded massages transferred between two computers via its noisy communication channels. The most important problem for effective signal transfer is to suppress the background noise. Therefore, for efficiency, information about the exchange signal to noise ratio (KO) is used. The noise ratio (KO) is the ratio of registered clicks induced by bio system signals to the background noise.

According to one embodiment herein, evolution of living species has made the information exchange by means of photon ratio/absorption optimal. Since the average rate of background radiation normally should be constant in time, then for a given bio-system with limited radiation intensity the optimal method to achieve a high KO level is to make most of the bio system radiation to be concentrated inside short time intervals i.e. radiation should be in the form of bursts with encoded signals transferred to other bio-systems.

According to one embodiment herein, in loach fish the egg colonies produced during the breeding have maximum survival achieved if all the eggs develop with same speed. External factors, such as water flow violate this condition. The bio photon signaling between the distant eggs of the same colony restores their simultaneous development. There is significant synchronization in the egg development. Optical contact between eggs of different ages hinders this synchronization and early eggs stop development, this proves photon signals of eggs have different structures which encodes characteristics about their age and corresponding development.

According to one embodiment herein, for the growth stimulation of cells with cellular electromagnetic waves/radiation three petri dishes are prepared with a standard growing culture of CD34. The first petri dish is labeled as “control”. Growth of cells is slow in control dish. Dish one is examined under the microscope after one week. The number of pulsatile cells is one to two per square centimeter. In the second petri dish the cells are stimulated by red light. Stimulation by red light is constant for one week. After one week the petri dish is examined under microscope. The observation reveals that the number of pulsatile cells is three to four per square centimeter. In the third petri dish the cells are subjected to photonic and electromagnetic waves of the cells. A special device is used to take the signal from the second petri dish to the cells in the third culture/petri dish. The cells in the third petri dish are constantly expose to electromagnetic and photonic waves. The microscopic examination of the third petri dish after one week shows a rate of growth of six to seven pulsatile cells per square centimeter.

The petri dish experiment shows that there is increased growth in the number of cells when stimulated by photonic and electromagnetic waves.

According to one embodiment herein, the difference in aging cells and young cells are evaluated based on the photonic and electromagnetic waves emitted. The epithelial cells are obtained from the foreskin of circumcised new born infant. The epithelial cells from the infant are compared to epithelial cells taken from upper thigh of 80 year old men. With a device it is noted the number of bursts of photons emitted from the newborn tissue are significantly higher than the photonic signals emitted from aging tissue. This assessment indicates the aging process reduces the vitality, energy and signals in an older person's cells.

According to one embodiment herein, two samples of endobronchial cancer cells are obtained. Sample one is untreated and examined for energy emission. It is observed that the bursts of energy are emitted.

Sample two is obtained after the patient is administered Photofrin II, 2.5 mgm/kg and 48 hours later treated by 630 nm laser light. This treated sample emitted only very erratic signals, mostly noise.

The evaluation indicates there is a time interval between the beginning of apoptosis, with slowing of signals, and then to complete disappearance of electromagnetic signals (stages of death). This finding is important in oncology and the treatment of cancers and abnormal cell growth.

According to one embodiment herein, the photonic cell signals are applied for diagnosis, therapeutic treatment, growth of cells, study of cell fusion and mitosis, detection of slow death in cellular structures, and scientific insight into the aging process with the possibility of manipulation to slow the process.

According to one embodiment herein, detection of cancer cells is based on the cell signals emitted even in the early stages of growth. The signals from a primary tumor stimulate metastasis to other organs. In the clinical setting it is important to detect primary and metastatic lesions early and begin therapeutic treatment as soon as possible. It is also postulated that analysis of cell signals indicate the effectiveness of various cancer treatments.

According to one embodiment herein, the cell signals contribute in understanding and treatment of cancer, aging process, and the cell fusion to form new tissues and organs.

Optical and ultraviolet excitation is released within the cell media, this is accomplished by quasi-particles called exciton. These particles are spread freely within the whole volume of the media. The excitations are strongly coupled with the electromagnetic field, therefore they effectively produced during photo absorption by the plants. The inverse process results in photon emission from biological media.

Excitons play an important role in the transfer of energy inside the cell, particularly during photosynthesis in plants and bacteria. Photon production by excitons is apparently non liner, for long distances of travel these photons should have solitonic properties. Exciton exchanges constitute effective signaling and affect.

The rate of biophoton emission is quite low at about 10 photons/cm²/second from the surface of a large bio system. In a non-coherent field of photons, it is called a stochastic assembly of photons. In stochastic assembly of photons, a single photon or narrow bunch of photons, detected by a bio system as a single click or one bit of information, is analogous to a standard photon detection device.

The same approach is applicable for exciton signaling produced or absorbed in a bio system. Under these assumptions the exciton signaling between two parts of the same bio system and photo signaling between two distant bio systems can be quite similar.

The signals which control cell mitosis and its functions, are similar to the standard binary encoded massages transferred between two computers via a noisy communication channel. The most important problem for effective signal transfer is to suppress the background noise. Therefore, for efficiency, information about the exchange signal to noise ratio (KO) is used. KO is the ratio of registered clicks induced by bio system signals to the background noise.

Evolution of living species has made the information exchange by means of photon ratio/absorption optimal. Since the average rate of background radiation normally should be constant in time, then for a given bio-system with limited radiation intensity the optimal method to achieve a high KO level is to make most of the bio system radiation to be concentrated inside short time intervals i.e. radiation should be in the form of bursts with encoded signals transferred to other bio-systems.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a schematic general representation of cell culture emitting bursts of energies/electromagnetic radiation for targeting cells, according to one embodiment herein.

FIG. 2 illustrates a graph indicating the binary quality of encoded signal bursts of energy from a growing cell, according to one embodiment herein.

FIG. 3 illustrates a graph indicating the growth of cells in petri dish one/control, according to one embodiment herein.

FIG. 4 illustrates a graph indicating the growth of cells in the second petri dish, wherein the cells are stimulated by red light constantly for one week, according to one embodiment herein.

FIG. 5 illustrates a graph indicating the growth of cells in the third petri dish, wherein the cells are exposed/stimulated by bursts of energies/signals from the second dish constantly for one week, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a system for targeting neoplastic cells without any side effects. Further the embodiments provide a simple method for targeting the neoplastic cells with electromagnetic radiation.

According to one embodiment herein, the method for stimulating a cell culture for growth with bursts of energies/cellular signals of cells, comprises of the following steps. The CD34 cells are cultured in three different culture plates comprising culture medium by following a standard culture protocol. The first culture plate is labelled as a control plate. The control plate comprises the CD 34 cells in culture medium. The first culture plate is incubated at 37° C. for one week. The first culture plate is not exposed to any bursts of energies/cellular signals of cells. The second culture plate comprising CD 34 cells in the culture medium is subjected to red light. The CD34 cells are stimulated by the red light constantly for one week. The second culture plate is incubated at 37° C. for one week. The third culture plate comprising CD 34 cells in a culture medium is subjected to plurality of bursts of energies/cellular signals of the CD34 cells in the second culture plate with device. The plurality of bursts of energies/cellular signals of the CD34 cells is detected and amplified by photo multiplier. The third culture plate is incubated at 37° C. for one week. The three culture plates comprising CD 34 cells are analyzed microscopically after one week.

According to one embodiment herein, the control culture plate illustrates 1-2 CD34 cells per square centimetre pulsatile. The second culture plate illustrates 3-4 CD34 cells per square centimetre pulsatile. The third culture plate illustrates 6-7 CD34 cells per square centimetre pulsatile. The photonic and electromagnetic waves increase a growth in number of cells.

According to one embodiment herein, the plurality of bursts of energies/cellular signals of the cells production is stipulated by biochemical reactions in the cells. The biophotons or bursts of energies/cellular signals of cells are produced by oxidization of proteins and acids in presence of oxygen in cell.

According to one embodiment herein, the cells producing bursts of energies/cellular signals of cells are in a state of growth. The bursts of energies/cellular signals of cells reach to nearby cells. The bursts of energies/cellular signals of cells increase a rate of division (mitosis) to a range. The range is 0-30%.

According to one embodiment herein, the transfer of the bursts of energies/cellular signals are binary in nature.

According to one embodiment herein, the bursts of energy are a summation of interior signals and membrane signals of cell organelles. The cell organelles produce action potential. The bursts of energy fall within an ultraviolet band of electromagnetic spectrum.

According to one embodiment herein, the method for evaluating aging in different cells with bursts of energies/cellular signals comprises the following steps. The epithelial cells from foreskin of circumcised new born infant are obtained. The cells from foreskin of the infant are cultured in laboratory following a standard protocol. The cells from upper thigh tissue of 80 year old individual are obtained. The cells from upper thigh tissue of 80 year old individual are cultured in laboratory following a standard protocol. The bursts of photons emitted from the cell culture of newborn foreskin tissue cells and upper thigh tissue of an individual are analyzed. The bursts of energies/cellular signals emitted from the new born foreskin tissue cells are more than that of the bursts of energies/cellular signals emitted from upper thigh tissue of an individual.

According to one embodiment herein, the method of analyzing photonic signals/bursts of energies/cellular signals emitted from apoptotic/cell undergoing cell death, comprises the following steps. Two endobronchial cancer cell sample are obtained from a patient. The first sample of the endobronchial cancer cell is obtained when the patient is not administered any chemical agent or a drug. The cells are cultured according to a standard protocol. The second sample of the endobronchial cancer cell are obtained after administering the patient with a photoferin II at a concentration of 2.5 mgm/Kg body weight. The endobronchial cancer cells are taken after 48 hours from a time of treating with a 630 nm laser light. The sample-2 cells undergo cell death/apoptosis.

According to one embodiment herein, the first sample emits photonic signals/bursts of energies/cellular signals. The second sample emits erratic signal or noise. The second sample cells indicate that the cells undergoing cell death do not produce photonic signals/bursts of energies/cellular signals.

According to one embodiment herein, the mechanism of biophoton production is stipulated by biochemical reactions in the cells. When the atoms of proteins and acids are oxidized in presence of oxygen the biophoton's are produced, and affect the other bio-systems.

According to one embodiment herein, when the cells are in the state of growth then the radiation emitted by the cells reaches to several centimeters. The radiations increase the rate of cell division (mitosis) upto 30% higher to the standard values.

According to one embodiment herein, the phenomenon of mitotic energy from a cell emits radiation to a level of 10⁴ more intense for cellular communication or bio-system communication in a state of apoptosis or stress. The mitogenic effect cannot be described within the standard framework of cellular and biological systems. The mitogenic effect is very similar to information exchange between two distant computers by binary encoded massages. The analysis of the signal exchange is done with a photomultiplier.

According to one embodiment herein, optical and ultraviolet excitation is released within the cell media, this is accomplished by quasi-particles called exciton. These excitons are spread freely within the whole volume of the culture media. The excitations are strongly coupled with the electromagnetic field, therefore the excitons are effectively produced during photo absorption by the plants. The inverse process results in photon emission from biological media.

According to one embodiment herein, excitons play an important role in the transfer of energy inside the cell, particularly during photosynthesis in plants and bacteria. Photon production by excitons is apparently non liner, for long distances of travel these photons should have solitonic properties. Exciton exchanges constitute effective signaling and affect plant growth.

According to one embodiment herein, the rate of biophoton emission is low at about 10 photons/cm²/second from the surface of a large bio system. The non-coherent field of photons is called as stochastic assembly of photons. In the stochastic assembly of photons a single photon or narrow bunch of photons, detected by a bio system as a single click or one bit of information, is analogous to a standard photon detection device.

According to one embodiment herein, the same approach is applicable for exciton signaling produced or absorbed in a bio system. Under these conditions the exciton signaling between two parts of the same bio system and photo signaling between two distant bio systems is similar.

According to one embodiment herein, the signals which control cell mitosis and cellular functions are similar to the standard binary encoded massages transferred between two computers via its noisy communication channels. The most important problem for effective signal transfer is to suppress the background noise. Therefore, for efficiency, information about the exchange signal to noise ratio (KO) is used. The noise ratio (KO) is the ratio of registered clicks induced by bio system signals to the background noise.

According to one embodiment herein, evolution of living species has made the information exchange by means of photon ratio/absorption optimal. Since the average rate of background radiation normally should be constant in time, then for a given bio-system with limited radiation intensity the optimal method to achieve a high KO level is to make most of the bio system radiation to be concentrated inside short time intervals i.e. radiation should be in the form of bursts with encoded signals transferred to other bio-systems.

According to one embodiment herein, in loach fish the egg colonies produced during the breeding have maximum survival achieved if all the eggs develop with the same speed. External factors, such as water flow violate this condition. The bio photon signaling between the distant eggs of the same colony restores their simultaneous development. There is significant synchronization in the egg development. Optical contact between eggs of different ages hinders this synchronization and early eggs stop development, this proves photon signals of eggs have different structures which encodes characteristics about their age and corresponding development.

According to one embodiment herein, the analysis of burst of energy from cells comprises of three phases.

### PHASE-I: Understanding the Cellular Emissions:

The animal and plant cells communicate with each other through electro-magnetic fields which fall into the ultraviolet (UV) band. The UV signals are weak, but at intervals come out as bursts of energy which are detectable. The UV signals are binary as in a computer. It is possible for these signals to travel several centimeters and they can be amplified for transmission for longer distances.

The cells grow or die based on absorption of photons, this is division in plant cells. The signals that cause mitosis (division & growth) are binary in characteristics. They are very weak and fall in noisy band, however from time to time the cells give out more powerful signals as bursts, or a sudden higher amplitude signals with a binary quality which we call bursts of energy which can travel several centimeters or meters and can affect other cells.

Cell signals are different from the action potential of cell membranes. The action potential in cardiac cells (myocytes) is ±70 milli-volts, depending on the influx of ions of K+(potassium) or Na (sodium) through the cell membrane. Binary cell signals are produced by burning acids with oxygen within the cells and are quite different from cell membrane action potential. These particles are called excitons and are strongly bound to the electro-magnetic field.

With sources such as mitochondria, or other sources to be determined, the sources of the action potential of progenitor cells are investigated and precise measuring of action potential with relation to organelles within the cells.

### PHASE-II: Analysis of Cellular Emissions and Effect of Cellular Emission on Cell Growth, Cell Death and Clinical Applications:

Phase II comprises analyzing and measuring of cellular radiation emission, particularly in CD4 or progenitor cells of bone marrow, and their relationship to the action potential of cell membranes. Following these measurements, the cell signals in healthy and diseased cells, in aging cells, in apoptosis and stimulated growth, and mitotic cells is analyzed. The cell signals are arrested or stopped by producing a negative harmonic for destruction of the cells (like cancer cells) by harmonics.

The signals are detected and amplified by a photomultiplier. The signals cause growth or demise of other cells, particularly cells with a quality of rapid mitosis (cancer cells) or death of these cells by negative harmonics of these signals.

The burst of energy is harvested from membrane of cells by a device. The burst of energy is the summation of interior and membrane signals and are different from the action potential. The burst of energy falls into the UV band of the electromagnetic spectrum.

Phase II also includes the analysis of the action potential of cell membrane myocytes which have three characteristics. The myocytes are either sinus, nodal (AV) or ventricular. To determine if the action potential of myocytes and progenitor cells is similar or different special probes, micro or nano voltmeters and oscilloscopes are used. The probes, micro or nano voltmeters and oscilloscopes also measure the action potential of mitochondria, golgi apparatus and other cell organelles. These measurements are compared in different electrolytic media.

The CD34 cells are good subjects for harnessing the bursts of energy in binary qualities as well as action potentials. The CD34 is a protein that in humans is encoded by the CD34 gene. The CD 34 is present in peripheral blood, in bone marrow, and cord blood. CD34 has pleuripotent characteristic. The CD 34 kits are available for laboratory culture example Stem Pro CD34. It is preferred to culture the CD34 cells in serum free medium. The assay is done by 0853 code HSEPCR kit which is available.

The action potential and the burst of energies are harnessed from membranes by photomultipliers.

Steps in Phase II are:
1. Study of the action potential of cell membranes
2. Measure the action potential with special probes, voltmeters, and oscilloscopes, and in different electrolytic media of:
   a. Mitochondria
   b. Golgi apparatus
   c. Other cell organelles
3. Detect cell signals (binary bursts) measurements, detect precise wavelengths, and record with photodetectors and nano-oscilloscopes
4. Stimulation of the growth phase in culture media, bioreactors, and measurement in young tissues and old tissues to evaluate the effect of the aging process
5. In the mitosis phase measure signals and action potential
6. In apoptosis and cell death phases (as in ischemic cells) measure signals and action potential
7. Determine the harmonics (advanced physics) of binary cell signals and reverse harmonics of the cells
8. Test the number of cell signals on mitosis, especially on cancer cells
9. Study effect of PDT (photodynamic therapy) and evaluate the signals and effect of reverse harmonics.

### PHASE-III: Application of the Cellular Radiation for Clinical Trials and Treatment:

The results and findings are reported and the techniques are subjected to encompass clinical applications.

### Example-1

### Analysis of Growth and Stimulation of Cells with Bursts of Energies

According to one embodiment herein, for the growth and stimulation of cells with bursts of energies three petri dishes are prepared with a standard growing culture of CD34. The first petri dish is labeled as “control”. The cellular growth is slow in control dish. Dish one is examined under the microscope after one week. The number of pulsatile cells is one to two per square centimeter.

In the second petri dish the cells are stimulated by red light. Stimulation of the cells by red light is constant for one week. After one week the petri dish is examined under microscope. The observation reveals that the number of pulsatile cells is three to four per square centimeter.

In the third petri dish the cells are subjected to photonic and electromagnetic waves of the cells. A special device is used to take the cellular signal (bursts of energies) from the second dish, put it in the third dish and constantly expose the cells in the third dish to electromagnetic and photonic waves. The microscopic examination of the third petri dish after one week shows a rate of growth of six to seven pulsatile cells per square centimeter.

The petri dish experiment shows that there is increased growth in the number of cells when stimulated by photonic and electromagnetic waves.

According to one embodiment herein, the photonic cell signals are applied for diagnosis, therapeutic treatment, growth of cells, study of cell fusion and mitosis, detection of slow death in cellular structures, and scientific insight into the aging process with the possibility of manipulation to slow the process.

According to one embodiment herein, detection of cancer cells is based on the cell signals emitted even in the early stages of growth. The signals from a primary tumor stimulate metastasis to other organs. In the clinical setting it is important to detect primary and metastatic lesions early and begin therapeutic treatment as soon as possible. It is also postulated that analysis of cell signals indicate the effectiveness of various cancer treatments.

According to one embodiment herein, the cell signals contribute in understanding and treatment of cancer, aging process, and the cell fusion to form new tissues and organs.

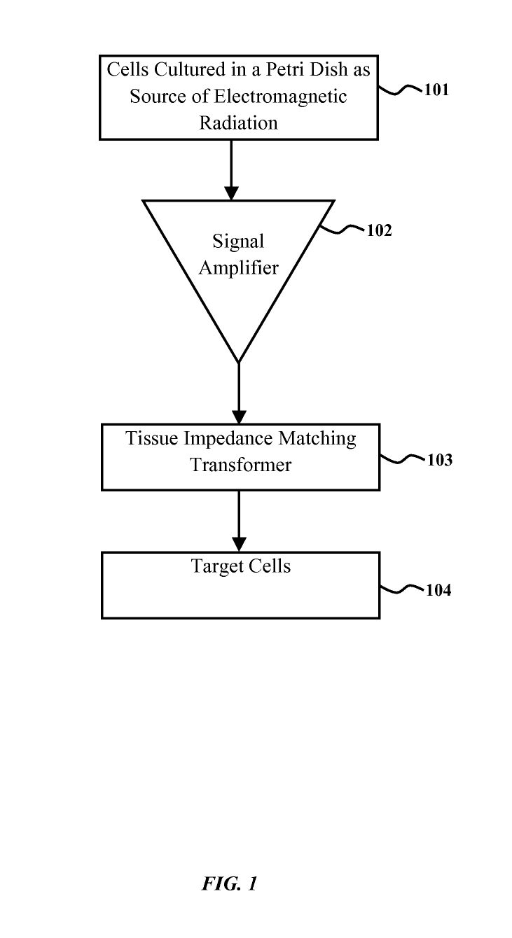

FIG. 1 illustrates a schematic general representation of cell culture emitting bursts of energies/electromagnetic radiation for targeting cells, according to one embodiment herein. The cells are cultured in a petridish, which act as the source of electromagnetic radiation or burst of energies (101). The electromagnetic signals or busts of energies are amplified by the signal amplifier device (102). The amplifier transmits the amplified electromagnetic signals or bursts of energies to a tissue impedance matching transformer (103). The target cells 104 or the cells to be subjected to electromagnetic radiation or bursts of energy.

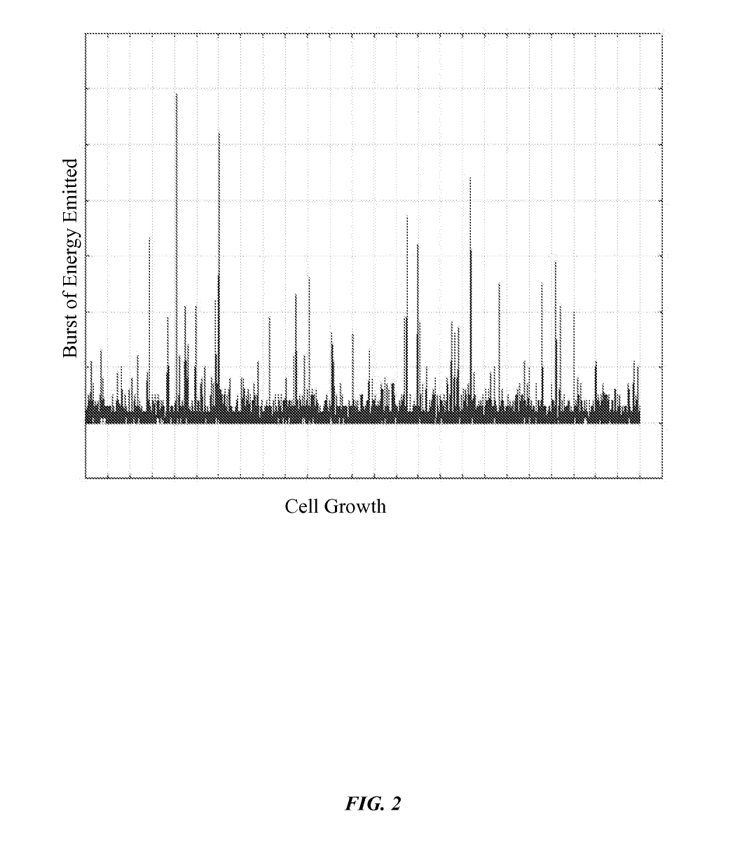

FIG. 2 illustrates a graph indicating the binary quality of encoded signal bursts of energy from a growing cell, according to one embodiment herein. The mitogenic effect is analyzed within the standard framework of cellular and biological systems. The bursts of energy are similar to information exchange between two distant computers by binary encoded massages. The analysis of these exchanges is done with a photomultiplier.

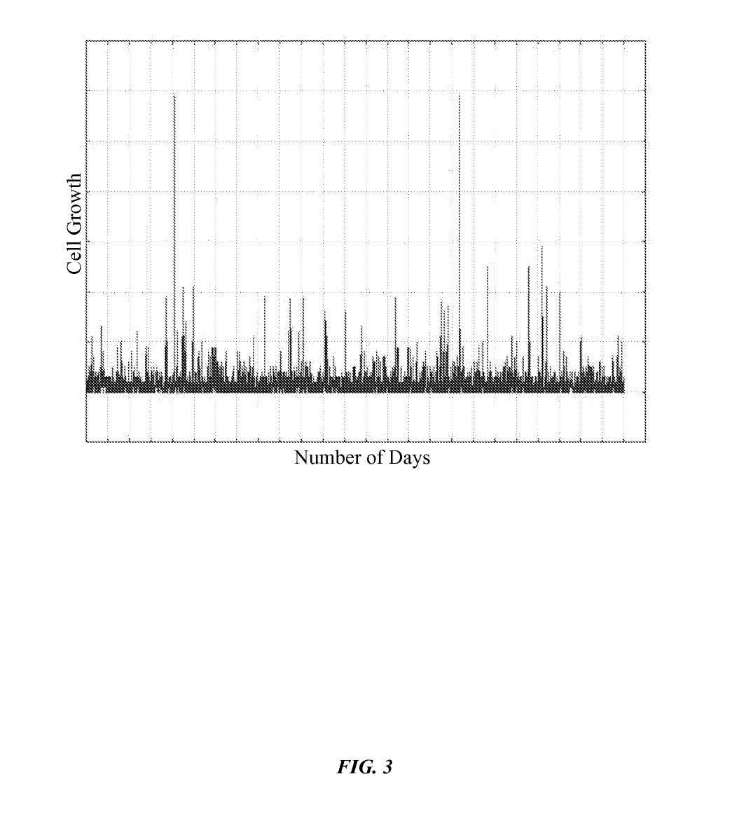

FIG. 3 is a graph illustrating the growth of cells in petri dish one/control, according to one embodiment herein. The CD34 cells are grown for one week with no stimulation. The growth of the cells is counted by microscopic examination. The cell growth is slow. Dish one is examined under the microscope after one week. The numbers of pulsatile cells are one to two per square centimeter.

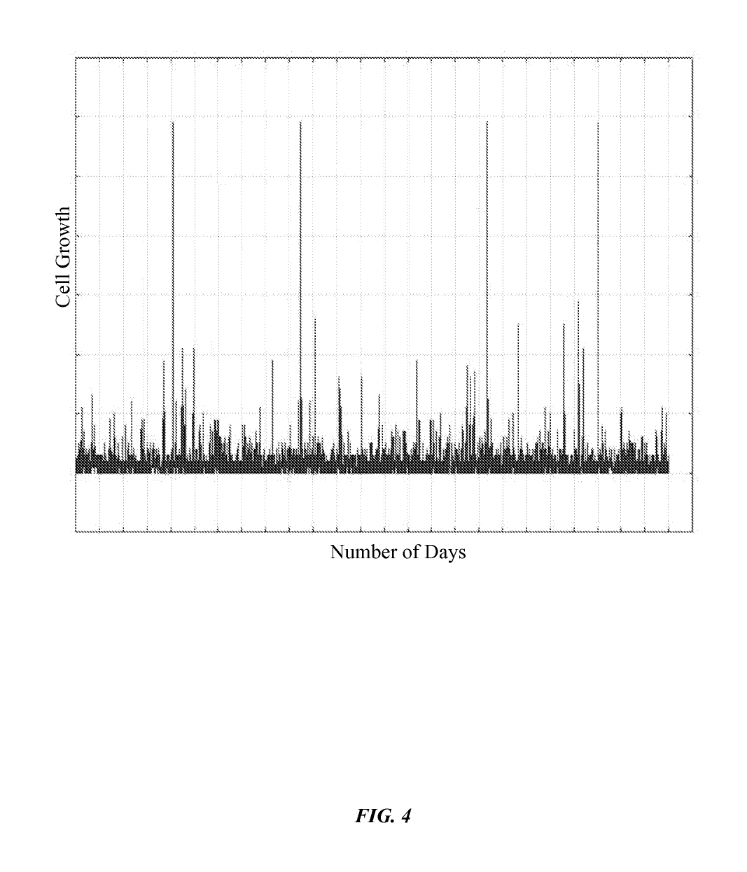

FIG. 4 is a graph illustrating the growth of cells in second petri dish, wherein the cells are stimulated by red light constantly for one week, according to one embodiment herein. In the second petri dish the CD34 cells are exposed to red light for one week and then counted by microscopic examination. The petridish is monitored under microscope. The number of pulsatile cells is three to four cells per square centimeter.

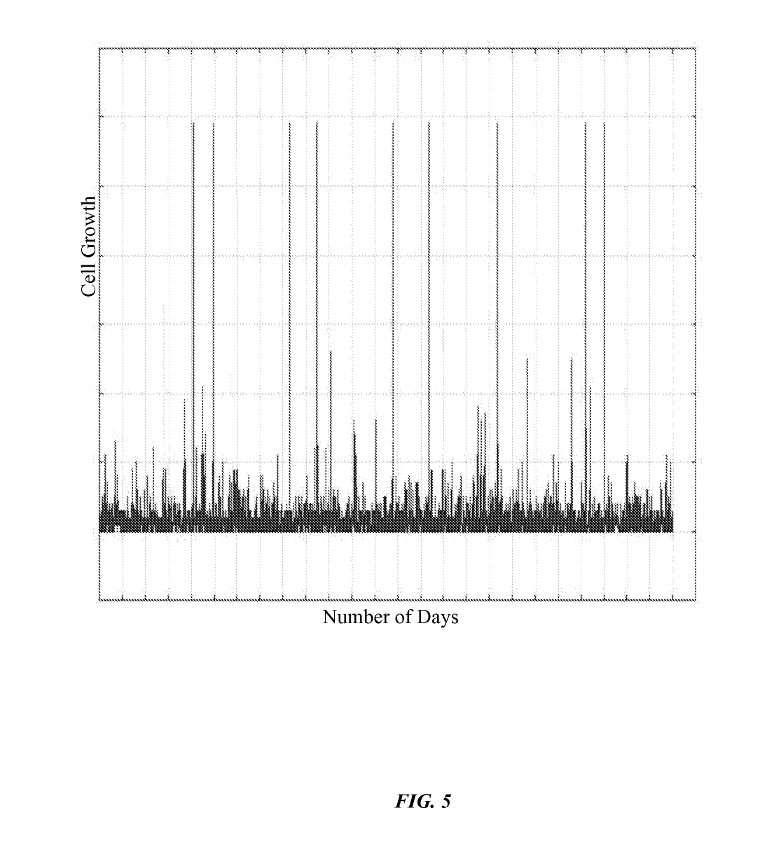

FIG. 5 is a graph illustrating the growth of cells in third petri dish, wherein the cells are exposed/stimulated by bursts of energies/signals from the second dish constantly for one week, according to one embodiment herein. A device is used to take the bursts of energies/signals from the second dish to the third dish and constantly expose the cells in the third dish to electromagnetic and photonic waves. The petri dish three-cells are exposed to photonic and electromagnet waves for one week and then counted by microscopic examination. Microscopic examination after one week shows a rate of growth of six to seven pulsatile cells per square centimeter.

### Example-2

### Analysis of Aging in Different Cells with Bursts of Energies

According to one embodiment herein, the difference in aging cells and young cells are evaluated. The epithelial cell obtained from the foreskin of circumcised new born infant is compared to epithelial cells taken from upper thigh of 80 year old men. With a device it is analyzed that the number of bursts of photons emitted from the newborn foreskin tissue are significantly higher than signals emitted from aging tissue. This assessment indicates the aging process reduces the vitality, energy and signals in an older person's cells.

### Example-3

### Analysis of Signals Emitted During Apoptosis and Cell Death

According to one embodiment herein, two samples of endobronchial cancer cells are obtained. Sample one is obtained when the patient is not treated with chemical agent or drug. Sample one is examined for energy emission. It is observed that there are bursts of energy emitted.

Sample two is obtained after the patient is administered Photofrin II, 2.5 mgm/kg IV and 48 hours later treated by 630 nm laser light. This treated sample emitted only very erratic signals, mostly noise.

The evaluation indicates there is a time interval between the beginning of apoptosis, with slowing of signals, and then to complete disappearance of electromagnetic signals (stages of death). This finding is important in oncology and the treatment of cancers and abnormal cell growth.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method for stimulating a cell culture for growth with bursts of energies/cellular signals of cells, the method comprises the steps of:
culturing CD34 cells in three different culture plates comprising a culture medium by following a standard culture protocol;
labelling a first culture plate as a control plate, and wherein the control plate comprises the CD 34 cells in a culture medium, and wherein the first culture plate is incubated at 37° C. for one week, and wherein the first culture plate is not exposed to any bursts of energies/cellular signals of cells;
subjecting a second culture plate comprising CD 34 cells in a culture medium to a red light, and wherein the CD34 cells are stimulated by the red light constantly for one week, and wherein the second culture plate is incubated at 37° C. for one week;
subjecting a third culture plate comprising CD 34 cells in a culture medium to a plurality of bursts of energies/cellular signals of the CD34 cells in the second culture plate with a device, and wherein the plurality of bursts of energies/cellular signals of the CD34 cells is detected and amplified by a photo multiplier, and wherein the third culture plate is incubated at 37° C. for one week; and
wherein the three culture plates comprising CD 34 cells are analyzed microscopically after one week.

2. The method according to claim 1, wherein the control culture plate illustrates 1-2 CD34 cells per square centimetre pulsatile.

3. The method according to claim 1, wherein the second culture plate illustrates 3-4 CD34 cells per square centimetre pulsatile.

4. The method according to claim 1, wherein the third culture plate illustrates 6-7 CD34 cells per square centimetre pulsatile, and wherein a photonic and electromagnetic waves increases a growth in number of cells.

5. The method according to claim 1, wherein the plurality of bursts of energies/cellular signals of the cells production is stipulated by biochemical reactions in the cells, and wherein biophotons or bursts of energies/cellular signals of cells are produced by an oxidization of proteins and acids in presence of oxygen in cell.

6. The method according to claim 1, wherein the cells producing bursts of energies/cellular signals of cells are in a state of growth, and wherein the bursts of energies/cellular signals of cells reaches to nearby cells, and wherein the bursts of energies/cellular signals of cells increases a rate of division (mitosis) to a range, and wherein the range is 0-30%.

7. The method according to claim 1, wherein a transfer the bursts of energies/cellular signals are binary in nature.

8. The method according to claim 1, wherein the bursts of energy is a summation of interior signals and membrane signals of cell organelles, and wherein the cell organelles produce action potential, and wherein the bursts of energy falls within an ultraviolet band of electromagnetic spectrum.

9. A method for evaluating aging in different cells with bursts of energies/cellular signals, the method comprises the steps of:
obtaining epithelial cells from foreskin of circumcised new born infant, and wherein the cells from foreskin of the infant are cultured in laboratory following a standard protocol;
obtaining the cells from upper thigh tissue of 80 year old individual and wherein the cells from upper thigh tissue of 80 year old individual are cultured in laboratory following a standard protocol; and
analyzing a bursts of photons emitted from the cell culture of newborn foreskin tissue cells and upper thigh tissue of an individual, and wherein the bursts of energies/cellular signals emitted from the new born foreskin tissue cells are more than that of the bursts of energies/cellular signals emitted from upper thigh tissue of an individual.

10. The method of analyzing photonic signals/bursts of energies/cellular signals emitted from apoptotic/cell undergoing cell death, the method comprises the steps of:
obtaining two endobronchial cancer cell sample from a patient;
obtaining a first sample of the endobronchial cancer cell when the patient is not administered any chemical agent or a drug, and wherein the cells are cultured according to a standard protocol;
obtaining a second sample of the endobronchial cancer cell after administering the patient with a photoferin II at a concentration of 2.5 mgm/Kg body weight, and wherein the endobronchial cancer cells are taken after 48 hours from a time of treating with a 630 nm laser light, and wherein the sample-2 cells undergo cell death/apoptosis.

11. The method according to claim 10, wherein the first sample emits photonic signals/bursts of energies/cellular signals.

12. The method according to claim 10, wherein the second sample emits erratic signal or noise, and wherein the second sample cells indicate that the cells undergoing cell death do not produce photonic signals/bursts of energies/cellular signals.
